(19) United States
(12) Patent Application Publication
(10) Pub. No.: US 2022/0409521 A1
(43) Pub. Date: Dec. 29, 2022

(54) COMPOSITION FOR COSMETICS

(71) Applicant: FE:I BEAUTY TECH, INC., New York, NY (US)

(72) Inventor: Xi Huang, Montvale, NJ (US)

(21) Appl. No.: 17/902,742

(22) Filed: Sep. 2, 2022

Related U.S. Application Data
(63) Continuation-in-part of application No. PCT/US2021/033755, filed on May 21, 2021.

(51) Int. Cl. A61K 8/98; A61K 8/19; A61K 8/02; A61Q 1/00; A61Q 17/00

(52) U.S. Cl. CPC A61K 8/987; A61K 8/19; A61K 8/0241; A61Q 1/00; A61Q 17/00; A61K 2800/412; A61K 2800/651; A61K 2800/43

(57) ABSTRACT

The present invention relates to a cosmetic for application to a user's skin. The cosmetic is provided with a colorant including iron oxides. The cosmetic also includes a high buffering-capacity agent configured to react with an external acidic source coming in contact with the cosmetic and to inhibit release of iron ions from the iron oxides.

### CROSS-REFERENCE TO RELATED APPLICATION

This is a continuation-in-part of International Patent Application No. PCT/US2021/033755 filed May 21, 2021, entitled COMPOSITION FOR COSMETICS, which claims priority to and the benefit of U.S. Provisional Patent Application No. 63/029,039, filed May 22, 2020, entitled COMPOSITIONS FOR COSMETICS, the disclosures of both of which are incorporated herein by reference in their entirety.

### FIELD OF THE INVENTION

The present invention relates to the fields of cosmetics, methods for making and using cosmetics, and methods for enhancing the appearance of a person's skin.

### BACKGROUND OF THE INVENTION

Many different cosmetic compositions have been developed in the past. The compositions described herein provide improved anti-aging effects for skin.

### SUMMARY OF THE INVENTION

In one embodiment, a high buffering-capacity (“HBC”) agent is incorporated into a cosmetic having iron oxides. In one embodiment, the cosmetic includes a composition in a hydrated form (e.g., composition contains at least enough liquid phase to provide a medium or mechanism for transferring ions between iron oxides and HBC agent such as an aqueous phase or a water- and/or oil-based emulsion). In one embodiment, the cosmetic includes a composition in a hydrated non-anhydrous form (e.g., Zinc sulfate heptahydrate (ZnSO₄.7H₂O)). In one embodiment, the cosmetic includes a composition in a hydrated anhydrous form (e.g., ethylene glycol (OHCH₂CH₂OH) or other glycols suitable for use in skincare product formulations). In one embodiment, the cosmetic includes a composition for a concealer, a foundation, powders, an eye shadow, a blush, a lipstick, a mineral makeup, a cream, a lotion, a serum, a sunscreen, a base, a mascara, a toner, a mask, and a milk. In one embodiment, the HBC agent includes at least one of pearl powder and an analog thereof (e.g., one with high alkaline cation from the second column of the periodic table (e.g., alkaline earth metals) but weak acid (e.g., pKa range from 3 to 6). In another embodiment, the HBC agent includes at least one of pearl powder, calcium carbonate, calcium citrate, calcium phosphate, calcium silicate, calcium molybdate, calcium tungstate, magnesium carbonate, magnesium phosphate, magnesium silicate, magnesium selenate, barium carbonate, barium phosphate, barium silicate, barium oxalate, barium molybdate, barium manganate, barium selenate, beryllium carbonate, beryllium phosphate, beryllium silicate, strontium carbonate, strontium phosphate, strontium silicate, strontium molybdate, strontium tungstate, strontium selenate, and a combination thereof.

In one embodiment, a colored cosmetic composition includes a colorant including iron oxides, and a high buffering-capacity (“HBC”) agent. In one embodiment, the HBC agent includes at least one of pearl powder and an analog thereof (e.g., one with high alkaline cation from the second column of the periodic table (e.g., alkaline earth metals) but weak acid (e.g., pKa range from 3 to 6)). In another embodiment, the high buffering capacity agent includes at least one of pearl powder, calcium carbonate, calcium citrate, calcium phosphate, calcium silicate, calcium molybdate, calcium tungstate, magnesium carbonate, magnesium phosphate, magnesium silicate, magnesium selenate, barium carbonate, barium phosphate, barium silicate, barium oxalate, barium molybdate, barium manganate, barium selenate, beryllium carbonate, beryllium phosphate, beryllium silicate, strontium carbonate, strontium phosphate, strontium silicate, strontium molybdate, strontium tungstate, strontium selenate, and a combination thereof.

The inventor herein has discovered, surprisingly and unexpectedly, that under an acidic condition, iron oxides in cosmetics can be solubilized and release “free” iron ions, which can cause damage to skin and premature aging. In one embodiment, when an external acidic material is introduced to the cosmetic applied to skin, the HBC agent is configured to maintain the pH of the cosmetic at or near neutral (i.e., pH 7), thereby inhibiting solubilization of the iron oxides contained in the cosmetic and release of “free” iron ions therefrom. In one embodiment, the external acidic material can be an endogenous source, such as the skin and/or sweat excreted therefrom, and/or an exogenous source, such acidic rain. The HBC agent therefore keeps the cosmetic safe for use by a user.

### BRIEF DESCRIPTION OF THE DRAWINGS

For a better understanding of the present invention, reference is made to the following detailed description of various exemplary embodiments considered in conjunction with the accompanying drawings, in which:

FIG. 1 shows color charts of synthetic iron oxides, namely, red, yellow and black, in mass tones and tint tones;

FIG. 2 shows the results of an example illustrating the effect of water soluble FeSO₄ on ferritin induction;

FIG. 3 shows the results of an example illustrating the increasing effect of acidified iron oxide red and yellow on ferritin induction;

FIG. 4 shows the results of an example illustrating the effect of acidification of a concealer on ferritin induction;

FIG. 5 shows the results of an example illustrating the buffering capacity of a concealer;

FIG. 6 shows the results of an example illustrating the effect of a concealer on ferritin induction in the absence of acidification;

FIG. 7 shows the results of an example illustrating the effects of varying amounts of pearl powder on the buffering capacity of a concealer;

FIG. 8 shows the results of an example illustrating the effect of pearl powder in a concealer on ferritin induction; and

FIG. 9 shows the results of an example illustrating the effect of pearl powder in a concealer on ferritin induction using various concealers to treat with human dermal fibroblast (HDF) cells.

FIG. 10 shows the results of an example illustrating the effect of pearl powder in a concealer on ferritin induction using treatment with HaCat cells.

### DETAILED DESCRIPTION OF EXEMPLARY EMBODIMENTS

Embodiments are now discussed in more detail referring to the drawings that accompany the present application. It is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, and some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; exemplary embodiments are provided merely to be illustrative. Among other things, for example, subject matter may be embodied as methods, devices, components, or systems. The following detailed description is, therefore, not intended to be taken in a limiting sense.

Throughout the specification and/or claims, terms may have nuanced meanings suggested or implied in context beyond an explicitly stated meaning. Likewise, the phrase “in one embodiment” as used herein does not necessarily refer to the same embodiment and the phrases “in another embodiment” and “other embodiments” as used herein do not necessarily refer to a different embodiment. It is intended, for example, that covered or claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

In one embodiment, a high buffering-capacity (“HBC”) agent is incorporated into a cosmetic containing iron oxides as a colorant. In one embodiment, the cosmetic includes a composition in a hydrated form (e.g., composition contains at least enough liquid phase to provide a medium or mechanism for transferring ions between iron oxides and HBC agent such as an aqueous phase or a water- and/or oil-based emulsion). In one embodiment, the cosmetic includes a composition in a hydrated non-anhydrous form (e.g., Zinc sulfate heptahydrate (ZnSO₄.7H₂O)). In one embodiment, the cosmetic includes a composition in a hydrated anhydrous form (e.g., ethylene glycol (OHCH₂CH₂OH) or other glycols suitable for use in skincare product formulations). In a further embodiment, the cosmetic includes a composition for a concealer, a foundation, powders, an eye shadow, a blush, a lipstick, a mineral makeup, a cream, a lotion, a serum, a sunscreen, a base, a mascara, a toner, a mask, or a milk. In another embodiment, the present invention is useful in any cosmetic containing iron oxides.

As used herein, the terms “high buffering-capacity agent”, “HBC agent”, and “buffering agent” means an agent, compound or material configured to react with an external acidic source or material and to inhibit (i.e., reduce iron ion release such that the amount of iron ion released from compositions with the buffering agent is less than the amount of iron ion released from compositions that do not include the buffering agent) release of iron ions from iron oxides in its associated cosmetic and/or to inhibit ferritin formation under acidic conditions. In one embodiment, a surface of particles of the HBC agent is hydrophilic. In one embodiment, the HBC agent is a non-diffusible (e.g., water insoluble) agent, compound, or material. In one embodiment, the HBC agent may reduce ferritin formation by about 0.2% or more. In another embodiment, the HBC agent may reduce ferritin formation by about 0.2% or less. In one embodiment, the HBC agent may reduce ferritin formation by about 30% or more. In another embodiment, the HBC agent may reduce ferritin formation by about 60% or less. The external acidic source or material is a biological and/or environment source or material that is not part of the cosmetic itself, but mixes or otherwise comes in contact with the cosmetic when it is applied to a user's skin. Examples of the external acidic material includes the user's skin, sweat, acid rain, an acidic beauty product, etc.

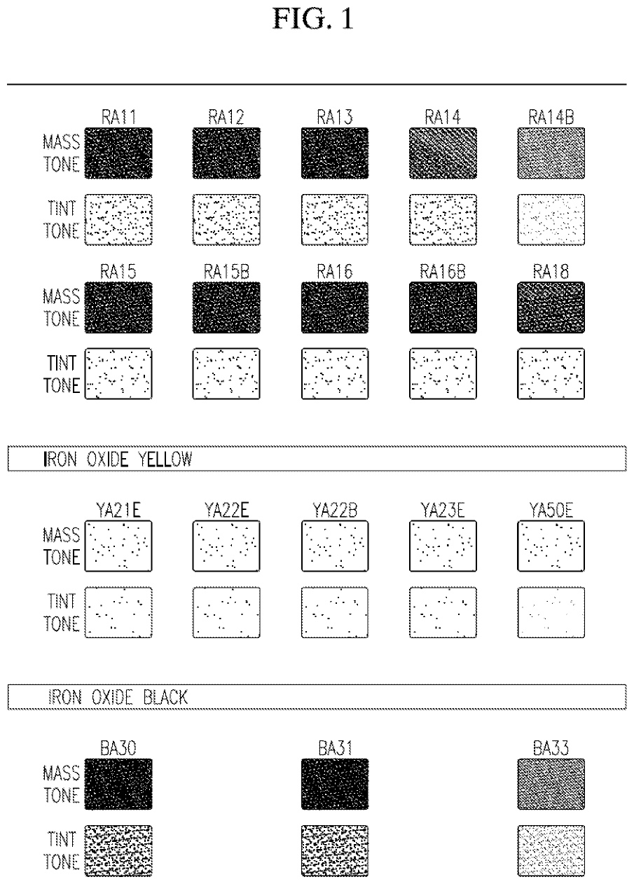

In cosmetics, iron-based colorants, for example, iron oxides, iron hydroxides and iron oxyhydroxides can be used. More particularly, iron oxides are the main pigments used for matching skin tones in foundations, powders, concealers, and other makeup for the face. Iron oxides can also be found in eye shadows, blushes, powders, lipstick, and mineral makeup. Generally, iron oxides are available in three basic shades: black (Fe₃O₄, CI 77499), yellow (FeOOH or Fe(OH)₃, CI 77492), and red (Fe₂O₃, CI 77491). By mixing them in different proportions, various color shades can be formed (see, e.g., FIG. 1).

The inventor herein has discovered, surprisingly and unexpectedly, that when subjected to an acidic condition, iron oxides in cosmetics can be solubilized and release “free” iron ions. As a result, conventional concealers, foundations and other cosmetics containing iron oxide colorants could cause damage to skin and pre-mature aging. The inventor herein has, surprisingly and unexpectedly, discovered that adding a buffer with a high buffering capacity can inhibit concealers from iron solubilization and oxidant formation.

In one embodiment, when the cosmetic is exposed to an external acidic material or source (e.g., when it is applied to a person's skin, or when it comes in contact with sweat or acidic rain), the HBC agent is configured to react with the external acidic material or source and to inhibit release of iron ions from iron oxides in the cosmetic. In one embodiment, the HBC agent is configured so as to interact with the external acid material or source and to neutralize same. In one embodiment, the HBC agent is configured to inhibit the external acidic material or source from significantly affecting (e.g., lowering more than 0.1 pH) the original pH of the cosmetic (e.g., the pH of the cosmetic prior to its exposure to the external acidic material or source or prior to its application to a person's skin). In one embodiment, the HBC agent is configured to inhibit the external acidic material or source and allows the composition to have a pH that remains substantially stable (e.g., the pH of the cosmetic prior to its exposure to the external acid material or source or prior to its application to a person's skin remains within one pH upon exposure to an external acidic or source). In one embodiment, the HBC agent is configured to react with the external acidic material or source and to maintain the original pH of the cosmetic substantially constant (e.g., within one pH of the original pH). In one embodiment, the HBC agent is configured to react with the external acidic source or material and to maintain the pH of the cosmetic substantially at neutral (e.g., pH range of about 6.5 to about 7.5). In one embodiment, the HBC agent is configured to react with the external acidic material or source and to maintain the pH of the cosmetic substantially at neutral (e.g., pH range of about 6.5 to about 7.5) or near neutral (e.g., within two pH of pH 7). In one embodiment, the HBC agent is configured to react with the external acidic material or source and to maintain the pH of the cosmetic between about 6.8 and about 7.2. In another embodiment, the HBC agent is configured to react with the external acidic material or source and to maintain the pH of the cosmetic between about 6.5 and about 8. In other embodiments, the HBC agent is configured to react with the external acidic material or source and to maintain the pH of the cosmetic between about 6 and about 10 or above 6.0.

In one embodiment, the HBC agent includes pearl powder and/or calcite powder. Pearl and calcite contain high levels of calcium carbonate (CaCO₃). In one embodiment, pearl powder may contain at least 90% calcium carbonate with the remaining percentage of the pearl powder including proteins, amino acids, and peptide. In another embodiment, the HBC agent may be any one of analogs of pearl or calcite powder (e.g., one with high alkaline cation from the second column of the periodic table (e.g., alkaline earth metals) but weak acid (e.g., pKa range from 3 to 6)). Non-limiting examples of such analogs include pearl powder, calcium carbonate, calcium citrate, calcium phosphate, calcium silicate, calcium molybdate, calcium tungstate, magnesium carbonate, magnesium phosphate, magnesium silicate, magnesium selenate, barium carbonate, barium phosphate, barium silicate, barium oxalate, barium molybdate, barium manganate, barium selenate, beryllium carbonate, beryllium phosphate, beryllium silicate, strontium carbonate, strontium phosphate, strontium silicate, strontium molybdate, strontium tungstate, strontium selenate, and a combination thereof. Pearl powder with a combination of calcium carbonate and amino acids, peptides, and proteins may be softer on skin than calcite and other HBC agents. Calcium-based HBC agents are more fine-tuned than pearl powder and calcite, and are particularly suitable because skin contains high levels of calcium ions. Additionally, dissolution of calcium carbonate will release calcium ion and carbon dioxide (CO₂), which has limited effects on the skin.

In one embodiment, the amount of the HBC agent included in the cosmetic ranges from about 0.01% (w/w) to about 10% (w/w) relative to the overall weight of the cosmetic. In another embodiment, the HBC agent amount ranges from about 2.0% (w/w) to about 10% (w/w). In yet another embodiment, the HBC agent amount ranges from about 0.5% (w/w) to about 5% (w/w). In a further embodiment, the HBC agent amount ranges from about 1% (w/w) to about 2% (w/w).

In one embodiment, the cosmetic can include conventional components that are included in a cosmetic product. Such components are readily understood by a person of ordinary skill in the art. Examples of such components are disclosed in U.S. Patent Application Publication Nos. 2010/0183528 A1 published Jul. 22, 2010; 2019/0105254 A1 published Apr. 11, 2019; and 2012/0269753 A1 published Oct. 25, 2012, the disclosures of all of which are incorporated herein by reference in their entireties.

In one embodiment, the HBC agent is in powder form. In one embodiment, the sizes of the particles of the HBC agent range from about 0.05 μm to about 30 μm. In one embodiment, the sizes of the particles of the HBC agent range from about 0.1 μm to about 30 μm. In one embodiment, the sizes of the particles of the HBC agent range from about 0.3 μm to about 20 μm. In one embodiment, the sizes of the particles of the HBC agent range from about 0.3 μm to about 15 μm. In one embodiment, the sizes of the particles of the HBC agent range from about 0.3 μm to about 10 μm. In one embodiment, the sizes of the particles of the HBC agent range from 0.3 μm to about 5 μm. In one embodiment, the sizes of the particles of the HBC agent range from about 0.3 μm to about 2 μm. In one embodiment, the sizes of the particles of the HBC agent range from about 0.03 μm to about 1 μm. In other embodiments, the sizes of the particles of the HBC agent are greater than about 0.05 μm, about 0.1 μm, or about 0.3 μm and/or less than about 30 μm, about 20 μm, about 15 μm, about 10 μm, or about 5 μm. In one embodiment, the HBC agent can be prepared in any method known in the art for preparing powder. For instance, pearls can be crushed, grinded or milled into fine powder using a conventional blender, grinder, mill, etc.

In one embodiment, the HBC agent is not treated with any agent. For example, the HBC agent is not surface-treated with any agent, such as hydrophobilizing agent, such that it is hydrophilic and can readily react with protons (H+). In one embodiment, the HBC agent is prepared as a metal ion absorbent by performing ion exchange. In one embodiment, the HBC agent is formed as fine particles having a large surface area for absorbing iron ions.

In one embodiment, the HBC agent is added to a cosmetic composition including iron oxides to form a desired cosmetic. In one embodiment, the cosmetic includes a composition in a hydrated form (e.g., composition contains at least enough liquid phase to provide a medium or mechanism for transferring ions between iron oxides and HBC agent such as an aqueous phase or a water- and/or oil-based emulsion). In one embodiment, the cosmetic includes a composition in a hydrated non-anhydrous form (e.g., Zinc sulfate heptahydrate (ZnSO₄.7H₂O)). In one embodiment, the cosmetic includes a composition in a hydrated anhydrous form (e.g., ethylene glycol (OHCH₂CH₂OH) or other glycols suitable for use in skincare product formulations). In one embodiment, the cosmetic includes a composition for a concealer, a foundation, powders, an eye shadow, a blush, a lipstick, a mineral makeup, a cream, a lotion, a serum, a sunscreen, a base, a mascara, a toner, a mask, or a milk. In one embodiment, the cosmetic composition can be made using any methods known in the art. For instance, a cosmetic preparation (e.g., an emulsion or aqueous form) containing iron oxides can be prepared in a conventional manner, and then the HBC agent can be added to and homogenized with the colorant preparation prior to performing finishing steps, such as an optional drying step, etc. In one embodiment, the HBC agent can be prepared in a hydrated phase (e.g., an aqueous phase or as an emulsion). For instance, the HBC agent can be prepared in an aqueous phase by pre-dispersing the HBC agent in an organic solvent, and then adding to an oil phase, such as pentylene glycol, before mixing with a water phase. In one embodiment, the HBC agent can be prepared as an emulsion by pre-dispersing the HBC agent in a water phase and then adding to an oil phase. In one embodiment, the HBC agent in an aqueous phase or as an emulsion is added to a cosmetic composition containing including iron oxides to form a desired cosmetic, such as a concealer, a foundation, powders, an eye shadow, a blush, a lipstick, a mineral makeup, a cream, a lotion, a serum, a sunscreen, a base, a mascara, a toner, a mask, and a milk, etc. In one embodiment, a cosmetic composition containing the HBC agent is prepared in an emulsion or aqueous form containing a solution such as water. In one embodiment, the HBC agent in an aqueous composition inhibits formation of iron ions, as discussed herein.

In one embodiment, the cosmetic composition is in a hydrated form (e.g., composition contains at least enough liquid phase to provide a medium or mechanism for transferring ions between iron oxides and HBC agent such as an aqueous phase or a water- and/or oil-based emulsion). In one embodiment, the cosmetic includes a composition in a hydrated non-anhydrous form (e.g., Zinc sulfate heptahydrate (ZnSO₄.7H₂O)). In one embodiment, the cosmetic includes a composition in a hydrated anhydrous form (e.g., ethylene glycol (OHCH₂CH₂OH) or other glycols suitable for use in skincare product formulations). In one embodiment, the hydrated form of the cosmetic composition includes a fluid that provides a medium or mechanism for transferring ions between the iron oxides and the HBC agent. The hydrated form improves the interaction between the iron oxide and the HBC agent and the buffering effects of the HBC agent. Also, increasing the water content of the hydrated form of the cosmetic composition can further improve the interaction between the iron oxide and the HBC agent and the buffering effects of the HBC agent. In addition, the HBC agent in a hydrated phase improves the uniformity of the HBC agent in the cosmetic composition (e.g., homogenizes). The homogenized composition has a pH that remains substantially stable (e.g., the pH of the cosmetic composition remains within one pH) when the cosmetic composition is in a container (e.g., a bottle containing the cosmetic composition). In one embodiment, the cosmetic composition is prepared such that its pH is and/or remains substantially at about 7. In one embodiment, the composition is prepared such that its pH is and/or remains at or above about 6.8. In one embodiment, the cosmetic composition provides a stable formulation for containing the HBC agent.

The substantially stable, homogenized composition inhibits the formation of foam in the cosmetic composition during storage in the container. For example, calcium carbonate can react with acids to produce CO₂ gas, creating foams in the formulation. In addition, the homogenous cosmetic composition spreads the HBC agent uniformly across a user's skin when the cosmetic composition is applied. Over time, the HBC agent remains uniformly spread across the user's skin, even if the liquid contents diminish due to evaporation. This improves the coverage of the HBC agent on the skin, which improves the protection against the release of iron ions caused by the external acidic sources or materials. Also, the homogenous cosmetic composition provides instant protection on the user's skin. The user's skin can produce acidic materials (e.g., protons (H+)) and the uniform application of the HBC agent is above to consume the acidic materials before the iron oxide because the HBC agent has a stronger alkalinity.

In one embodiment, the HBC agent is configured such that when the cosmetic composition is applied to a user's skin, the HBC agent is not absorbed into the skin, but remains on same to react with acidic sources or materials. In one embodiment, the HBC agent is water-insoluble and hence non-diffusible such that it remains on (i.e., is not diffused into) the skin to provide long-lasting protection against acidic sources or materials.

Provided below are a more detailed discussion of the discoveries made by the inventor herein and various examples associated with same.

Natural iron oxides, such as those from mines, often contain toxic metals, e.g., lead, arsenic, mercury, antimony and selenium, and are not therefore suitable for cosmetics even after undergoing substantial purification. As a result, iron oxides have been made in laboratories to assure their purity. Because synthetic iron oxides contain low concentrations of toxic metals, they have generally been known to be gentle. Synthetic iron oxides are considered as non-irritating to the skin and are not known to be allergenic. In general, it has been believed that iron oxides (e.g., CI 77499, CI 77492, CI 77491) used in cosmetic products are non-toxic and, thus, safe.

Despite the foregoing conventional belief, the inventor herein has discovered, surprisingly and unexpected, that synthetic iron oxides, even at its purest form (100% pure), could cause skin damage, and their use on skin may be undesirable. This is because iron is a good transition metal capable of producing reactive oxygen species (ROS), such as hydroxyl radicals (OH), through Haber-Weiss and Fenton reactions, as shown below:

Reaction(A):Fe³⁺+O₂″→Fe²⁺′+O₂(Haber-Weiss reaction)

Reaction(B):Fe²⁺+H₂O₂→Fe³⁺+OH⁻+OH(Fenton reaction)

Reaction(C):H₂O₂+O₂⁻→OH⁻+OH+O₂(Net,iron as a catalyst)

ROS is known to have toxic effects on cell metabolism and aging, including damages to DNA, RNA, proteins, lipids, oxidative deactivation of enzymes, alteration of signaling pathways, etc.

The surfaces of iron oxides are typically treated to make them hydrophobic, thereby making them resistant to solubilization. The inventor herein has discovered, surprisingly and unexpectedly, that such surface treatment is not effective in preventing iron oxides from solubilization under certain circumstances. More particularly, iron oxides can be solubilized and then become bioavailable after their topical application on the skin and/or when subjected to an acidic condition. For instance, the pH of the skin and the sweat is acidic. The inventor has discovered that iron oxides in cosmetics, such as concealers, can be solubilized and release potentially toxic “free” iron ions under acidic conditions. This solubilization is more likely in the presence of biological molecules, such as citrate, and under environmental stress, such as acid rains and sun exposure. According to reaction (C) above, iron ions act as a catalyst, and even a small amount of iron ions can cause formation of ROS for a long time (see also U.S. Patent Publication No. 2015/0024016 A1 published Jan. 22, 2015, the disclosure of which is incorporated herein by reference in its entirety).

### Example 1

The enhancing effect of water soluble ferrous sulfate (FeSO₄) on ferritin induction was examined. In this example, a ferritin was used as a biomarker for iron's bioavailability after incubation. Ferritin is an iron storage protein with the capacity of binding up to about 4,500 atoms of iron per molecule of ferritin.

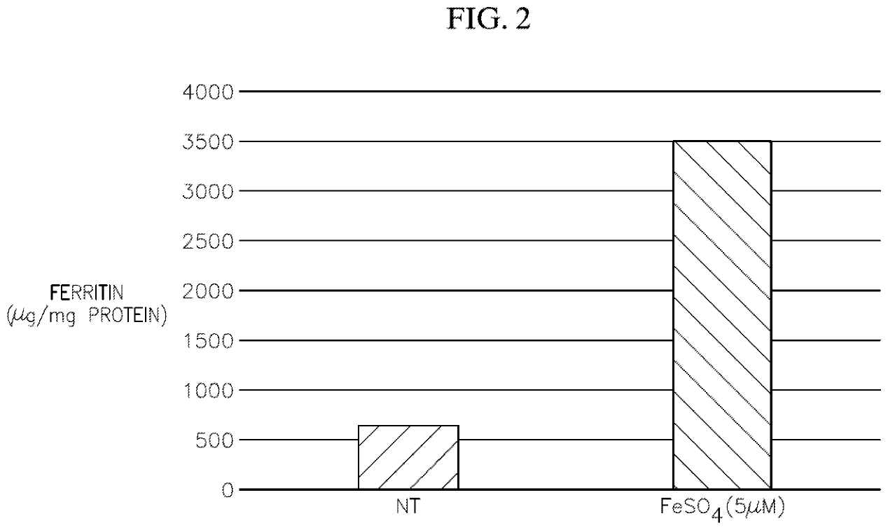

Primary human dermal fibroblast (HDF) cells were cultured in Dulbecco's modified eagle medium (DMEM) with GlutaMAX™ and 10% Fetal Bovine Serum (FBS) and 1% Streptomycin (antibiotics). Before treatment, cells were seeded in a 12-well plate (1 ml culture media and surface area of the well is approximately 4.0 cm² per well) overnight. After washing twice with cold phosphate buffered saline (PBS), HDF cells were starved in 0.1% FBS for overnight. Subsequently, cells were treated with ferrous sulfate septahydrate (FeSO₄·7H₂O) at a final concentration of 5 μM for additional 18 hours. Cells with no treatment were used as a control. Cells were then washed with PBS and lysed. Levels of ferritin and protein were measured by using commercially available enzyme-linked immunosorbent assays (ELISA) kits and quantitated with standard curves constructed with human liver ferritin and human serum albumin as standards. The results were expressed as μg ferritin per mg protein and are show in FIG. 2.

With reference to FIG. 2, a 5 μM concentration of ferrous sulfate (FeSO₄, fully water soluble and, thus, fully bioavailable) in the tissue culture media increased the level of ferritin by 570% in HDF cells, compared to the control. Assuming that this iron is fully deposited on the monolayer of the cells, it would be equivalent to 0.07 mg Fe²⁺/cm² (5 μM Fe²⁺ in 1 ml culture media with 4 cm² surface area in a 6-well plate). Previous studies have shown that ferritin is a pro-oxidant and is readily degraded by UVA when exposed to the sun, causing oxidant formation and matrix metalloproteinase-1 increase. Therefore, ferritin is a strong indicator of skin aging and photo-aging.

### Example 2

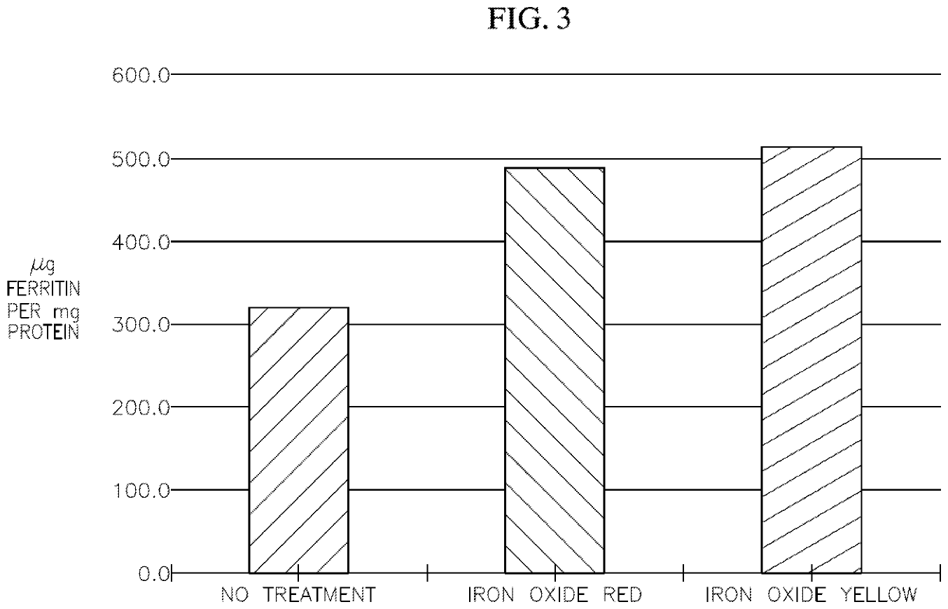

To show that iron oxides may release bioavailable iron when subjected to an acidic condition, the following two iron oxides were tested in a tissue culture system: iron oxide C177491 jojoba ester red (Fe₂O₃), and iron oxide C177492 jojoba ester yellow (Fe(OH)₃). The two iron oxides were premixed homogeneously in a 50% (v/v) of water and tertraethylene glycol and incubated at 3 mg/ml overnight at room temperature in a sodium acetate buffer (10 mM, pH 5.0), mimicking the pHs of acid rain or sweat. After overnight incubation (approximately 16 hours), the suspensions of iron oxides were used to treat HDF at 10 μg/cm² for 18 hours. The μg/cm² unit was used because iron oxides are not water soluble and, once they are added to the tissue culture media, they would be deposited on the monolayer of the cells. This 10 μg/cm² dose is believed to be less than the amount that is usually applied on the skin. After the foregoing treatment, cells were collected and levels of ferritin and proteins in cells were measured and calculated as described in Example 1. FeSO₄ was used as a positive control, and its associated data are not shown in FIG. 3, but its induction by FeSO₄ is comparable to that shown in FIG. 2. FIG. 3 shows the ferritin levels of the treated cells, as well as that of cells with no treatment (control). As shown in FIG. 3, the iron oxides red and yellow suspended in a weak acidic pH increased ferritin formation by 52.8% (488.4 ng/mg vs 319.7 ng/mg protein in the control groups) and 60.4% (512.9 ng/mg vs 319.7 ng/mg protein groups), respectively. The foregoing results indicate that iron is released from water insoluble iron oxides under a weak acidic condition, causing increased ferritin formation.

### Example 3

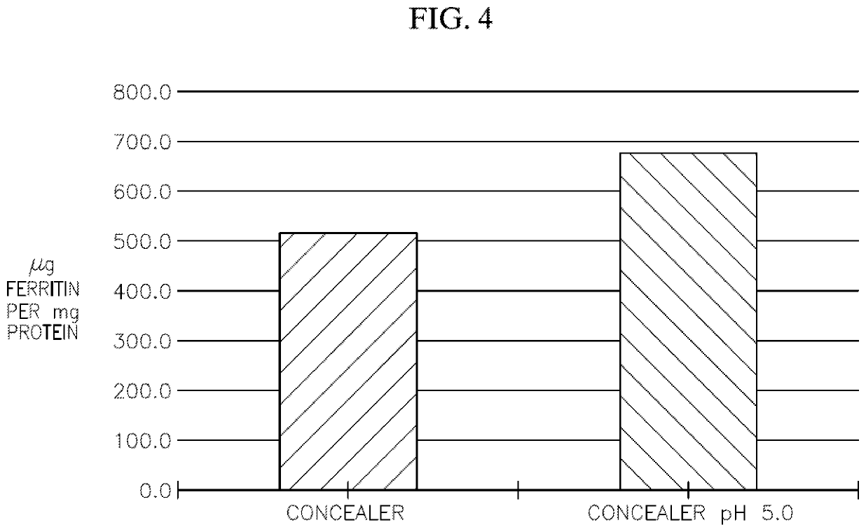

To further demonstrate that iron oxides incorporated in a cosmetic product can also increase ferritin formation, a concealer from Tarte™ Cosmetics containing 3.88% (w/w) yellow iron oxide, 1% (w/w) red iron oxide, and 0.42% (w/w) black iron oxide is premixed in water and tertraethylene glycol, incubated in an acidic acetate buffer (pH 5), and then treated with HDF cells as described in Example 2 above. After treatment, cells were collected, and the levels of ferritin and proteins in the cells were measured and calculated as described above in Example 1. A concealer sample with no acidic treatment was used as a control. As shown in FIG. 4, the concealer, which contained approximately a total of 5% of all three iron oxides or 0.5 mg/cm² dose and was pretreated in a slightly acidic environment as described above, showed an increase in ferritin by 30.7% as compared to the control. The foregoing results indicate that concealers containing iron oxides in a formulation could have greater effects on releasing bioavailable iron into cells, potentially causing harmful effects on the skin.

The hydrolysis constant (Ksp) of iron oxides could be low. However, iron oxides' Ksp may increase by several orders of magnitude with decreasing crystal size and acidic pH.

Based on the results of the foregoing studies, it is believed that surface treatment by jojoba ester or formulation in oil or in an emulsion (oil in water or water in oil) may not effectively prevent iron oxide acid solubilization. In the skin structure, cell membranes have lipid-and water-soluble components. Small particle sizes and lipid soluble iron oxides can penetrate readily into skin, and the acidic pH of the skin can make iron oxides solubilized and become bioavailable for oxidant formation.

### Example 4

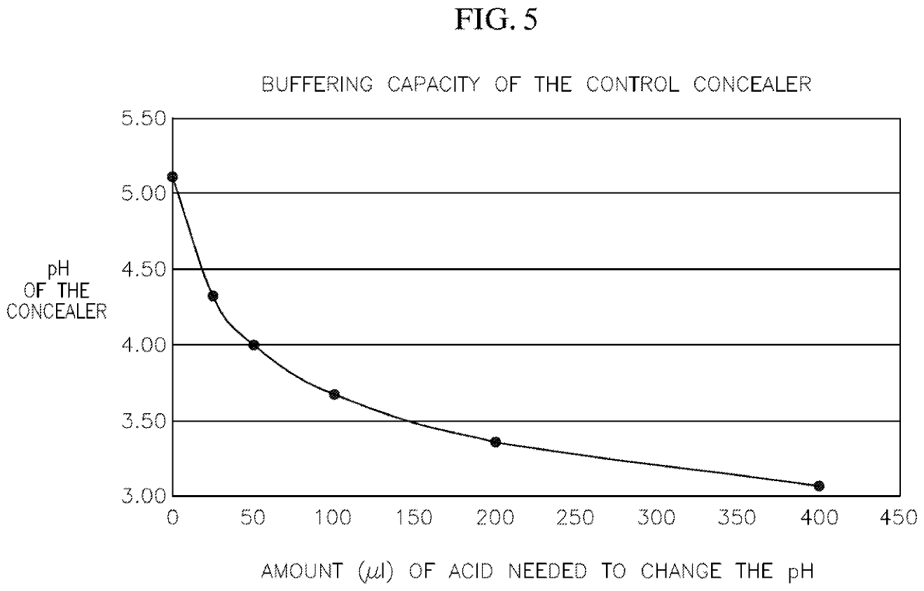

As shown above, the inventor herein has discovered that iron oxides are easily solubilized under weak acidic conditions. In this example, the buffering capacity of the concealer described in Example 3 was examined. The concealer was suspended in a 50% (v/v) of water and pentaethylene glycol at 3 mg/ml concentration. The initial pH of the suspension was recorded. Subsequently, 25 μl of 10 mM hydrochloric acid (HCl) was added to the suspension. After mixing thoroughly through shaking and vertexing for at least a minute, the pH of the suspension was recorded and plotted with the amounts of additional acid added. FIG. 5 shows that the pH of the suspension decreased dramatically as HCl was added. In particular, the initial pH of the concealer was 5.1. With the addition of 25 μl of 10 mM HCl, the pH dropped to 4.4, while the addition of additional 25 μl HCl (or a total of 50 μl HCl) caused the pH to further drop to 4.0. These results indicate that the concealer lacks protection against acidic pH environments, whether they are from an endogenous source, such as the skin or sweat, or from an exogenous source, such as acid rain. Under these conditions, iron oxides in the concealers can be solubilized and release “free” iron ions to cause oxidation and oxidative damage to the skin.

### Example 5

The effect of a concealer without acidification on ferritin induction was examined. The concealer described in Example 3 above was premixed in water and tertraethylene glycol and then treated with HDF cells as described in Example 2 above, but without acidification. After treatment, cells were collected, and the levels of ferritin and proteins in the cells were measured and calculated as described in Example 1 above. HDF cells without the concealer were used as a control.

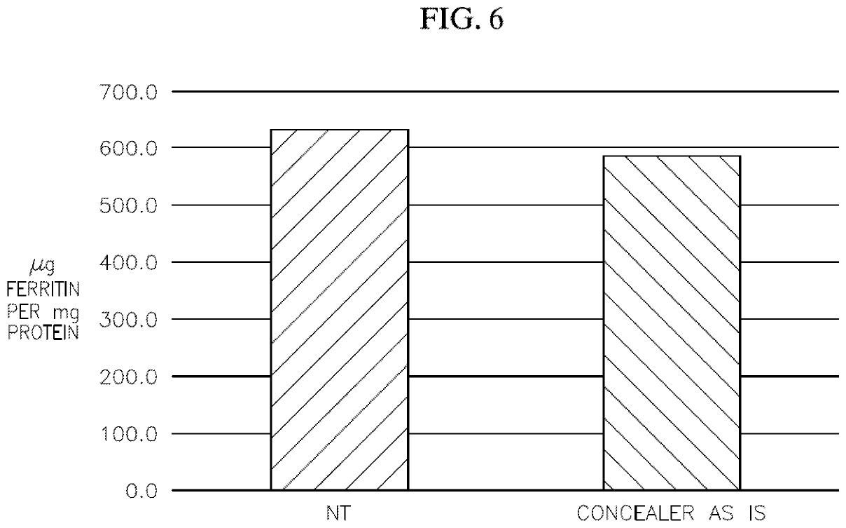

FIG. 6 shows that the levels of ferritin in the concealer treated cells are a little lower (585.0 ng ferritin/mg protein) than the non-treated cells (630.3 ng/mg). These results suggest the reason why iron oxide-containing concealers or other cosmetics have been considered safe. However, these results are due to the artifact of preset experimental conditions and do not actually reflect real-life pH conditions under which concealers and other cosmetics are used (e.g., on the skin which is acidic). More particularly, the pH of the cell culture media in this example was 7.4, which naturally inhibits iron acid solubilization, thereby causing no significant increase in ferritin levels. Because of this artifact, synthetic iron oxides have been considered safe for use on the skin. However, because iron oxides can be solubilized in real-life situations (e.g., when exposed to an acidic environment), they may not be as safe as they have been believed to be.

### Example 6

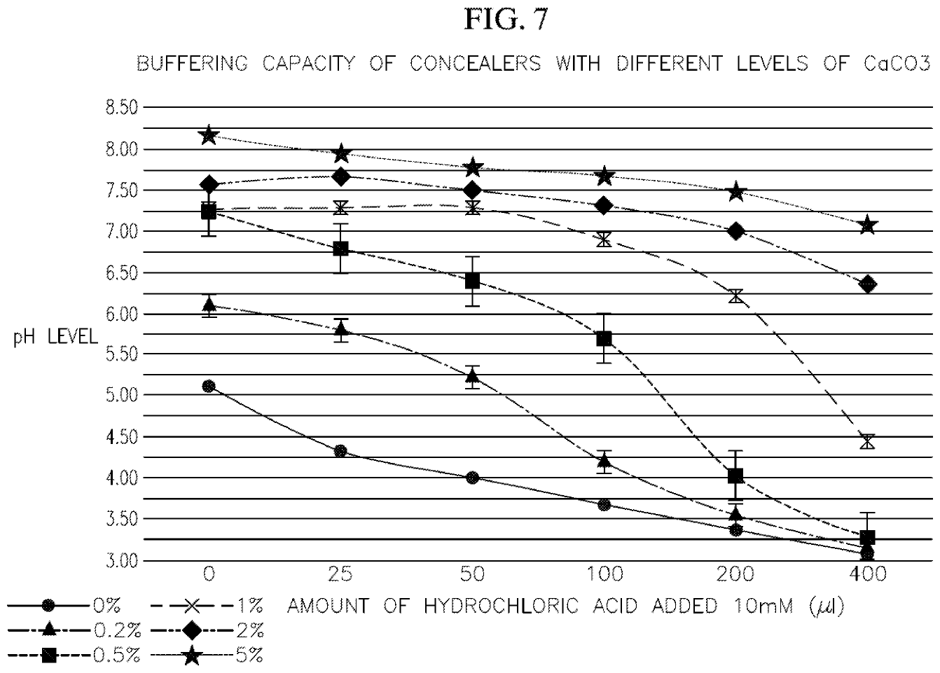

Conventional pearls were ground using a commercial mill from Jet Pulverizer to prepare pearl powder (a mean size of 2.03 μm, a median size of 1.53 μm, mode size of 1.41 μm, standard deviation of 1.72 μm, diameters on cumulative (%) as follows: 10%: 0.82 μm, 50% 1.53 μm, 90%: 3.69 μm, 99%: 9.80 μm, and 100% 17.36 μm). The pearl powder was then added to the concealer described in Example 3 above in the following amounts: 0.2%, 0.5%, 1%, 2%, 5% and 10% (w/w). A concealer sample without pearl powder is used as control. The concealer was then suspended in a 50% (v/v) of water and tertraethylene glycol at 3 mg/ml concentration, and the initial pHs of the suspensions were recorded. Subsequently, 25 μl of 10 mM HCl was sequentially added to each of the suspensions, mixing thoroughly through shaking and vertexing for at least a minute. The pHs of the suspensions were recorded and plotted in FIG. 7 when each amount of acid was added to same.

As shown in FIG. 7, the initial pHs of the suspensions with 0.2%, 0.5%, 1%, 2%, and 5% pearl powder were 6.1, 7.1, 7.2, 7.5 and 8.1 respectively. The initial pH of the suspension with 10% pearl powder was close to 9, its buffering capacity was extremely high, and its pH did not change significantly when HCl was added (not shown in FIG. 7). On the other hand, the initial pH of the control without pearl powder was 5.1. After adding 25 μl of 10 mM HCl, the pH levels dropped to 5.8, 6.8, 7.2, 7.5, and 7.9 in the samples with 0.2%, 0.5%, 1%, 2%, and 5% pearl powder, while the pH of the control dropped to 4.4. The drop in pH levels for 0%, 0.2%, 0.5%, 1%, 2%, and 5% was −0.7, −0.3, −0.3, 0, 0, and −0.2, respectively. With an additional amount of 25 μl HCl or a total amount of 50 μl HCl, the pHs were lowered to 5.3, 6.4, 7.2, 7.5, and 7.6 for the concealers with increasing orders of pearl powder. The pH of the concealer without pearl powder further dropped to 4.0. The decreases in pHs for 0%, 0.2%, 0.5%, 1%, 2%, and 5% were −1.1, −0.8, −0.7, 0, 0, and −0.5, respectively, when compared to their respective initial pHs. These results indicate that the samples containing 1% and 2% (w/w) pearl powder were able to maintain its original pH level after being exposed to an environmental and/or physiologically relevant amount of acid (e.g., from about 25 μl to about 50 μl of HCl).

### Example 7

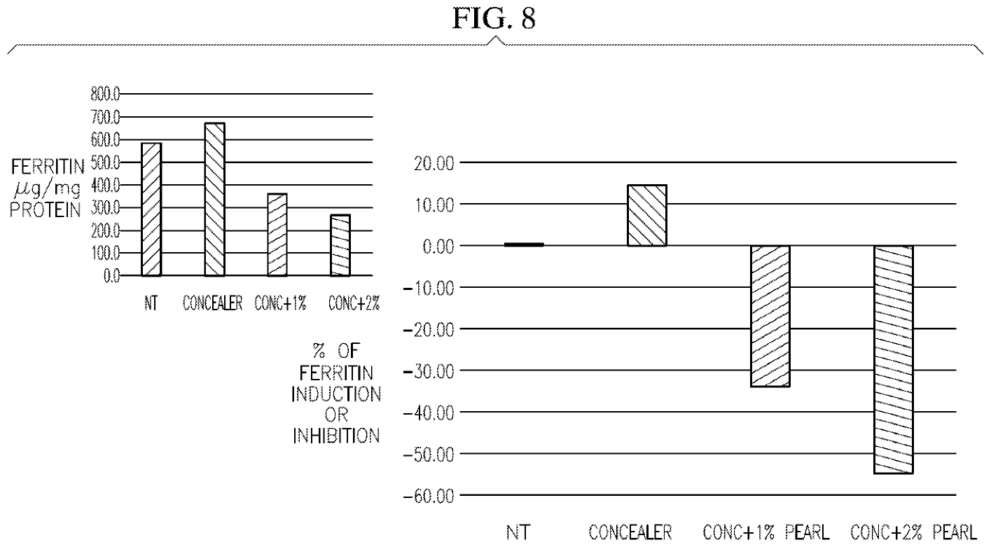

To determine whether concealers containing pearl powder inhibit ferritin formation under acidic conditions, samples of the concealer described in Example 3 above were mixed with 1% (w/w), and 2% (w/w) of the pearl powder described in Example 6. The mixtures were then mixed in water and tertraethylene glycol, incubated in acetate buffer (pH 5) overnight and then treated with HDF cells as described in Example 3 (i.e., at 10 μg/cm² for 18 hours). A concealer sample without pearl powder was used a reference, and no treatment was used as a control. After treatment, cells were collected, and the levels of ferritin and proteins in the cells were measured and calculated as described in Example 1 above. FIG. 8 shows that the concealer sample without pearl powder increased ferritin formation by 14.9% as compared to the control (no treatment). The concealer samples containing 1% and 2% pearl powder inhibited ferritin by 33.8% and 54.7%, respectively.

### Example 8

Two commercially available concealers/foundations were examined to determine whether concealers/foundations containing iron oxides without the high buffering agent disclosed herein will release iron ions similar to the control concealer. One concealer examined is IT Bye Under Eye (full coverage anti-aging water proof concealer, Tan Sand 31). The ingredient list of the IT Bye Under Eye (IT) concealer is provided below:
Caprylic/Capric Triglyceride, Bis-Diglyceryl Polyacyladipate-2, Vp/Hexadecene Copolymer, Cetyl Alcohol, Silica Dimethyl Silylate/Silica Dimethyl Silylate, Microcrystalline Wax, Phenoxyethanol, Water, Niacinamide, Sodium Hyaluronate, Magnesium Ascorbyl Phosphate, Tocopheryl Acetate, Ascorbyl Palmitate, Retinyl Palmitate, Pentaerythrityl Tetra-Di-T-Butyl Hydroxyhydrocinnamate, Hydrolyzed Collagen, Cholesteryl Isostearate, Cholesteryl Chloride, Cholesteryl Nonanoate, Glycerin, Steareth-20, Tocopherol, Silica, Chlorhexidine Digluconate, N-Hydroxysuccinimide, Benzoic Acid, BHT, Potassium Sorbate, Palmitoyl Tripeptide-1, Chrysin, Palmitoyl Tetrapeptide-7. May Contain (+/−): Titanium Dioxide (Ci 77891), Iron Oxides (Ci 77491, Ci 77492, Ci 7749).

As shown by the ingredient list, the IT concealer formulation is more lipophilic.

The other concealer examined is Estee Lauder Double Wear Stay-in-place Flawless Wear Concealer (3C Medium). The ingredient list of the Estee Lauder Double Wear Stay-in-place Flawless Wear Concealer (Estee Lauder) is provided below:
Water\Aqua\Eau, Cyclopentasiloxane, Trimethylsiloxysilicate, Phenyl Trimethicone, Butylene Glycol, Boron Nitride, Sorbitan Sesquioleate, Peg/Ppg-18/18 Dimethicone, Tribehenin, Magnesium Sulfate, Tocopheryl Acetate, Sodium Hyaluronate, Ethylhexylglycerin, Dimethicone, Methicone, Laureth-7, Glycerin, Cetyl Peg/Ppg-10/1 Dimethicone, Pentaerythrityl Tetra-Di-T-Butyl Hydroxyhydrocinnamate, Xanthan Gum, Alumina, Trisiloxane, Dimethicone Silylate, Sorbic Acid, Phenoxyethanol, Chlorphenesin, [+/−Iron Oxides (Ci 77491, Ci 77492, Ci 77499), Titanium Dioxide (Ci 77891), Mica]<ILN38896>

As shown by the ingredient list, the Estee Lauder formulation is more hydrophilic.

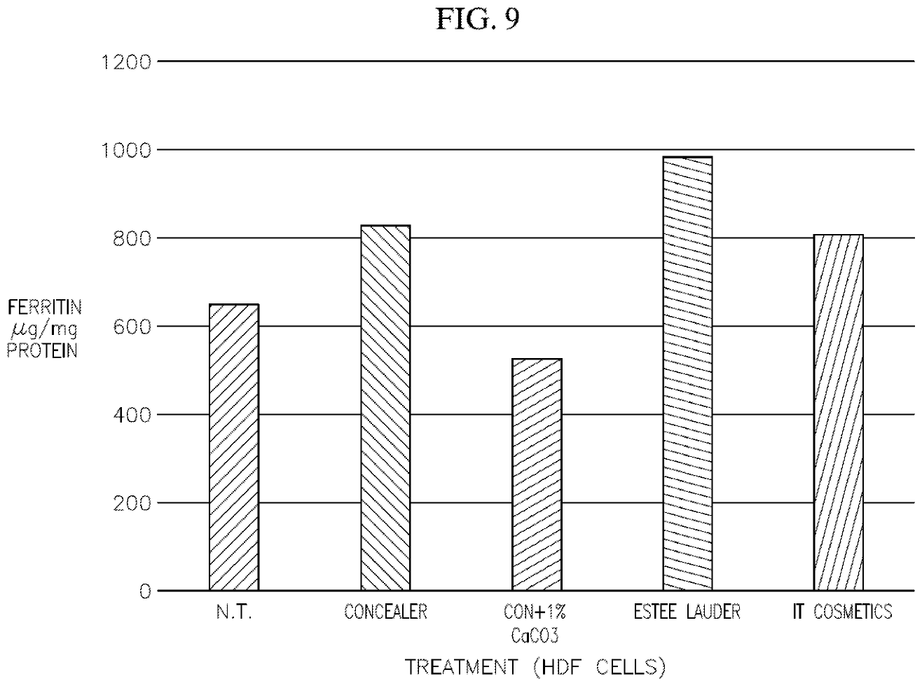

To determine whether the IT and Estee Lauder concealers promote ferritin formation under acidic conditions, samples of the IT and Estee Lauder concealers were premixed in water and tertraethylene glycol and incubated in an acidic acetate buffer having a pH of 5. The concealers were then treated with human dermal fibroblast (HDF) cells as described in Example 3 (i.e., at 10 μg/cm² for 18 hours). After treatment, the HDF cells were collected and the levels of ferritin and proteins in the HDF cells were measured and calculated as described in Example 1 above. The results were expressed as μg ferritin per mg protein and are show in FIG. 9.

Two other concealers, which were previously used in our examples, were used as references (e.g., reference concealers). One reference concealer included samples of the concealer described in Example 3 mixed with 1% (w/w) of the pearl powder described in Example 6. The other reference concealer included samples of the concealer described in Example 3 without pearl powder. Samples of the two reference concealers were premixed in water and tertraethylene glycol and incubated in an acidic acetate buffer having a pH of 5. The reference concealers were then treated with human dermal fibroblast (HDF) cells as described in Example 3 (i.e., at 10 μg/cm² for 18 hours). After treatment, the HDF cells were collected and the levels of ferritin and proteins in the HDF cells were measured and calculated as described in Example 1 above. The results were expressed as μg ferritin per mg protein and are show in FIG. 9.

A fifth concealer including samples of the concealer described in Example 3 with no treatment was used as a control (e.g., control concealer).

FIG. 9 shows that the concealer sample without pearl powder increased ferritin formation by 26.6% as compared to the control concealer (no treatment). Additionally, concealers from Estée Lauder and It Cosmetics also increased ferritin by 50.5% and 23.6%, respectively. In contrast, the reference concealer samples containing 1% pearl powder inhibited ferritin by 19.5%. These results indicate that these commercially available concealers have limited protection in releasing free iron ions to the skin.

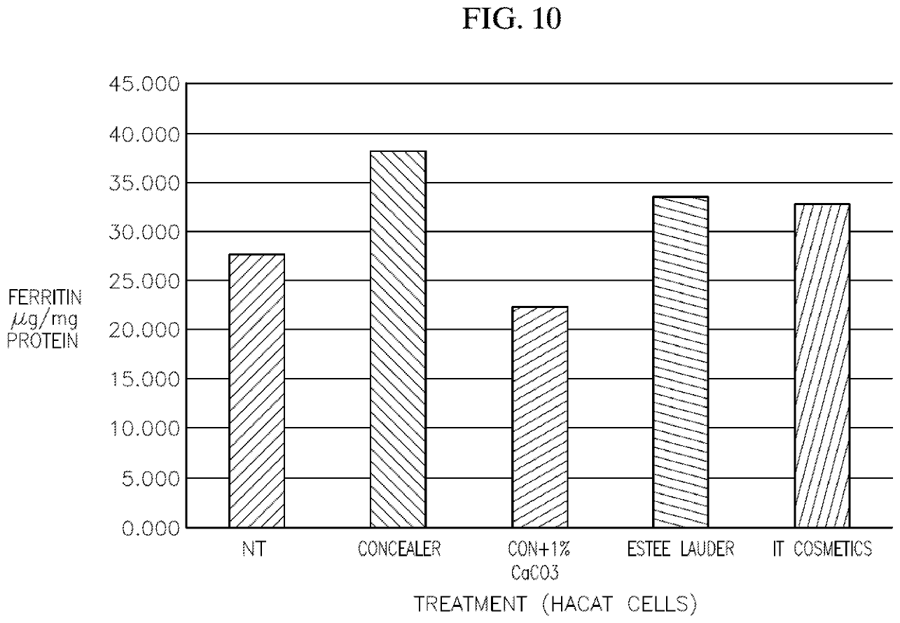

Subsequently, HaCat cells, which include an immortalized human epidermal keratinocytes cell line, were used for the same treatment conditions as described above. The results were expressed as μg ferritin per mg protein and are show in FIG. 10. It is shown that the background level of ferritin in HaCat cells (27.5 μg/mg protein) (FIG. 10) is much lower as compared to that of the primary HDF (651.4 μg/mg protein) (FIG. 9). However, the same pattern remains. The concealer sample without pearl powder increased ferritin formation by 38.8% as compared to the control (no treatment). Additionally, concealers from Estée Lauder and It Cosmetics also increased ferritin by 21.5% and 18.8%, respectively. In contrast, the concealer samples containing 1% pearl powder inhibited ferritin by 19.2%. These results indicate that these commercially available concealers have no protection in releasing free iron ions to the skin. The same holds true in the skin keratinocytes HaCat cells.

It will be understood by those having ordinary skill in the art and possession of the present disclosure that the embodiments described herein are merely exemplary in nature and that a person skilled in the art may make many variations and modifications thereto without departing from the scope of the present invention. All such variations and modifications are intended to be included within the scope of the invention.

I claim:

1. A method for inhibiting damage caused to a user's skin by a cosmetic containing one or more iron-based colorants, said method comprising the step of:
applying a cosmetic composition in a hydrated form to the user's skin, wherein the cosmetic composition comprises a colorant, which includes iron oxides, and a buffering agent, the buffering agent reacting with an external acidic source coming in contact with the cosmetic composition and inhibiting release of iron ions from the iron oxides.

2. The method of claim 1, wherein the buffering agent reacts with the external acidic source such that the pH level of the cosmetic composition remains substantially stable despite exposure to the external acidic source.

3. The method of claim 2, wherein the buffering agent reacts with the external acidic source and maintains the pH level of the cosmetic composition substantially at neutral.

4. The method of claim 1, wherein the pH level of the cosmetic composition is at or above about 6.8.

5. The method of claim 1, wherein the buffering agent includes at least one of pearl powder, calcium carbonate, calcium citrate, calcium phosphate, calcium silicate, calcium molybate, calcium tungstate, magnesium carbonate, magnesium phosphate, magnesium silicate, magnesium selenate, barium carbonate, barium phosphate, barium silicate, barium oxalate, barium molybate, barium manganate, barium selenate, beryllium carbonate, beryllium phosphate, beryllium silicate, strontium carbonate, strontium phosphate, strontium silicate, strontium molybate, strontium tungstate, strontium selenate, or a combination thereof.

6. The method of claim 1, wherein said buffering agent is prepared in a hydrated form.

7. The method of claim 1, wherein the buffering agent is included in the cosmetic in an amount ranging from about 0.01% (w/w) to about 10% w/w).

8. The method of claim 7, wherein the amount of the buffering agent ranges from about 0.01% (w/w) to about 2% (w/w).

9. The method of claim 1, wherein the buffering agent includes pearl powder having a size ranging from about 0.05 μM to about 30 μm.

10. The method of claim 1, wherein the cosmetic includes one of a lotion, a cream, a serum, a sunscreen, a base, an eye shadow, a lipstick, a mascara, a blush, a foundation, a concealer, and a mineral makeup.

11. A method for inhibiting damage caused to a user's skin by a cosmetic containing one or more iron-based colorants, said method comprising the step of:
preparing a cosmetic composition having a colorant, which includes iron oxides, wherein said preparing step includes the step of including in said cosmetic composition a buffering agent configured to react with an external acidic source coming in contact with the cosmetic composition and inhibiting release of iron ions from the iron oxides.

12. The method of claim 11, wherein the buffering agent reacts with the external acidic source such that the pH level of the cosmetic composition remains substantially stable despite exposure to the external acidic source.

13. The method of claim 12, wherein the buffering agent reacts with the external acidic source and maintains the pH level of the cosmetic composition substantially at neutral.

14. The method of claim 11, wherein the pH level of the cosmetic composition is at or above about 6.8.

15. The method of claim 11, wherein the buffering agent includes at least one of pearl powder, calcium carbonate, calcium citrate, calcium phosphate, calcium silicate, calcium molybate, calcium tungstate, magnesium carbonate, magnesium phosphate, magnesium silicate, magnesium selenate, barium carbonate, barium phosphate, barium silicate, barium oxalate, barium molybate, barium manganate, barium selenate, beryllium carbonate, beryllium phosphate, beryllium silicate, strontium carbonate, strontium phosphate, strontium silicate, strontium molybate, strontium tungstate, strontium selenate, or a combination thereof.

16. The method of claim 11, wherein said buffering agent is prepared in a hydrated form.

17. The method of claim 11, wherein the buffering agent is included in the cosmetic in an amount ranging from about 0.01% (w/w to about 10% (w/w).

18. The method of claim 17, wherein the amount of the buffeting agent ranges from about 0.01% (w/w) to about 2% (w/w).

19. The method of claim 11, wherein the buffering agent includes pearl powder having a size ranging from about 0.05 μm to about 30 μm.

20. The method of claim 11, wherein the cosmetic includes one of a lotion, a cream, a serum, a sunscreen, a base, an eye shadow, a lipstick, a mascara, a blush, a foundation, a concealer, and a mineral makeup.
